(19) United States
(12) Patent Application Publication
(10) Pub. No.: US 2001/0049366 A1
(43) Pub. Date: Dec. 6, 2001

(54) Topical solution formulations containing an antibiotic and a corticosteroid

(75) Inventors: Onkar N. Singh, Arlington, TX (US); Haresh G. Bhagat, Fort Worth, TX (US)

Correspondence Address: Alcon Universal Ltd., c/o Alcon Research, Ltd., R&D Counsel (Q-148), 6201 South Freeway, Forth Worth, TX, 76134-2099, US

(73) Assignee: Alcon Universal Ltd.

(21) Appl. No.: 09/779,366

(22) Filed: Feb. 8, 2001

Related U.S. Application Data
(60) Provisional application No. 60/181,317, filed on Feb. 9, 2000.

(51) Int. Cl. A61K 31/56; A61K 31/497

(52) U.S. Cl. 514/171; 514/253.08

(57) ABSTRACT

Solution formulations containing a corticosteroid, an antibiotic and a vitamin E tocopheryl derivative as a solubilizing agent are disclosed. The formulations are intended for topical application to the eye, ear, nose or skin.

This application claims priority to now abandoned U.S. Provisional Application, Ser. No. 60/181,317, filed February 9, 2000.

### BACKGROUND OF THE INVENTION

This invention relates to topically administrable solution formulations containing an antibiotic, a corticosteroid and a solubilizing agent.

Both solution and suspension compositions containing dexamethasone as the sole active agent are marketed. The solution compositions contain dexamethasone in the form of dexamethasone sodium phosphate. The suspension formulations contain dexamethasone in the form of dexamethasone alcohol. See Ophthalmic Drug Facts '99, Facts and Comparisons, St. Louis, Mo. (1999), p. 87. Additionally, aqueous anti-inflammatory/anti-infective combination products containing dexamethasone are currently marketed. See Ophthalmic Drug Facts '99, Facts and Comparisons, St. Louis, Mo. (1999), p. 121-122. The only such combination product identified as a solution is a neomycin sulfate/dexamethasone sodium phosphate solution product.

Spanish Patent Application No. 2,065,846 A1 (Feb. 16, 1995) discloses topically administrable ophthalmic and otic antibiotic/steroid combination products. Examples 1-3 illustrate ophthalmic suspension formulations containing certain drug combinations with excipients including nonionic polymers and nonionic surfactants. Example 1 is a formulation of clobetasone and lomefloxacin that contains a nonionic tonicity agent (glycerin). Example 2 is a formulation of fluoromethalone and norfloxacin that contains an ionic tonicity agent (sodium chloride). Example 3 is a formulation of ciprofloxacin and dexamethasone that contains a nonionic tonicity agent (mannitol).

U.S. Pat. Nos. 5,540,930 and 5,747,061 disclose topically administrable steroid suspension formulations that contain a nonionic polymer, a nonionic surfactant and a nonionic tonicity agent. The patents are directed toward “stable suspensions of water-insoluble steroid drugs of particle sizes ≦15 μm, which remain in such a state so as to allow for immediate suspension, when desired, even after extended periods of settling” (see the '061 patent's Abstract). The patents are based on a finding that “[u]nexpectedly, common tonicity agents such as aqueous solutions containing 0.9% NaCl, 0.1% EDTA, or phosphate buffer, even in concentrations as low as 1 mM, can not be employed to provide stable aqueous suspensions of corticosteroids such as [loteprednol etabonate (LE)]”('061 patent, Col. 2, lines 52-56).

The '061 patent is aimed at formulations that solved a need for “aqueous suspensions of corticosteroids such as LE which can be formulated without agglomeration” (Col. 2, lines 57-59). The '061 patent's formulations contain (A) a soft steroid such as LE present as particles preferably having a mean diameter of less than about 15 microns, (B) a nonionic polymer as a suspending agent, (C) a nonionic surfactant and (D) a nonionic tonicity agent. The '061 patent defines a “soft” drug as a biologically active chemical component characterized by predictable in vivo metabolism to non-toxic derivatives after it provides its therapeutic effect. The '061 patent teaches that “[i]t is essential that these components (A)-(D) be nonionic insofar as possible since it has now been discovered that the presence of ions is the major cause of caking” (Col. 3, lines 51-53). Nonionic diols such as glycerin or mannitol “rather than the commonly used sodium chloride” are identified as the preferred tonicity agents (see Col. 3, lines 53-56). The nonionic tonicity agent is preferably present in an amount of about 0.5 to 10% by weight.

U.S. Pat. No. 5,886,030 discloses the use of a vitamin E tocopheryl derivative, such as d-alpha tocopheryl polyoxyethylene glycol (1000) succinate (also known as “Vitamin E TPGS” or “tocophersolan”), to increase the comfort of topically administrable ophthalmic compositions. The vitamin E tocopheryl derivative also increases the solubility of poorly soluble ophthalmic agents in aqueous compositions. Dexamethasone is listed as the sole active ingredient in one of the formulations identified in Example 1 of the '030 patent.

### SUMMARY OF THE INVENTION

The present invention provides solution formulations of an antibiotic and a corticosteroid. The corticosteroid ingredient is not sufficiently soluble in water at desired levels without the aid of a solubilizing agent. For example, in the case where the corticosteroid is dexamethasone, the corticosteroid ingredient is not dexamethasone phosphate, a water-soluble form of dexamethasone, but would be dexamethasone alcohol or dexamethasone acetate. The solubilizing agent in the formulations of the present invention is a vitamin E tocopheryl derivative.

Among other factors, the present is based on the finding that solution formulations containing a fluoroquinolone antibiotic, dexamethasone alcohol and a vitamin E tocopheryl derivative as a solubilizing agent provide superior anti-inflammatory efficacy than formulations containing a fluoroquinolone antibiotic and dexamethasone phosphate.

### DETAILED DESCRIPTION OF THE INVENTION

Unless indicated otherwise, all ingredient amounts presented as a percentage are in weight/volume units, % (w/v).

Many ophthalmically and otically acceptable corticosteroids are known. The preferred corticosteroid ingredient of the present invention is dexamethasone alcohol or dexamethasone acetate, collectively referred to as “dexamethasone.” The antibiotic ingredient may be any water-soluble ophthalmically or otically acceptable antibiotic, including without limitation quinolone antibiotics. Although 6 des-F quinolones are suitable, fluoroquinolones are preferred. Examples of fluoroquinolones include, but are not limited to ciprofloxacin, moxifloxacin, gatifloxacin, gemifloxacin, norfloxacin, ofloxacin, levofloxacin and trovofloxacin. The antibiotic can be present in any ophthalmically or otically acceptable form such that it is in solution in the final formulation. A preferred fluoroquinolone antibiotic is ciprofloxacin. A preferred form of ciprofloxacin is ciprofloxacin hydrochloride, monohydrate.

The corticosteroid ingredient will comprise about 0.01-1% and the antibiotic ingredient will comprise about 0.1-1.5% of the formulations of the present invention. In the case where the corticosteroid is dexamethasone and the antibiotic is ciprofloxacin, the preferred amounts of dexamethasone and ciprofloxacin in the formulations of the present invention are 0.1% and 0.3%, respectively. In the case where the antibiotic is moxifloxacin, the preferred amounts of dexamethasone and moxifloxacin in the formulations of the present invention are 0.1% and 0.5%, respectively.

In addition to a corticosteroid and an antibiotic, the formulations of the present invention contain a vitamin E tocopheryl derivative. The vitamin E tocopheryl derivatives useful in the compositions of the present invention are highly water-soluble polyoxyalkylene glycol esters of vitamin E tocopheryl esters of a dicarboxylic acid. Representative esters of this type include the polyoxyethylene glycol esters of vitamin E tocopheryl esters of a dicarboxylic acid wherein the polyoxyethylene glycol moiety of the ester has a molecular weight in the range from about 600 to about 6000, preferably in the range from about 600 to about 1500. The most preferred ester is d-alpha tocopheryl polyoxyethylene glycol (1000) succinate, a polyoxyethylene glycol ester of α-tocopheryl succinate wherein the polyoxyethylene glycol moiety of the molecule has an average molecular weight of about 1000.

The compositions of the present invention will contain an amount of vitamin E tocopheryl derivative sufficient to solubilize the corticosteroid ingredient. In general, the amount of such solublizer in the compositions of the present invention will range from 0.5 to 20%. When the corticosteroid ingredient is present at a concentration of 0.1%, the vitamin E tocopheryl derivative is present at a concentration of at least 5% and preferably about 7%.

In addition to the active agents and the vitamin E tocopheryl derivative, the formulations of the present invention contain an ionic or nonionic tonicity agent; however, certain ionic tonicity agents, such as NaCl, if used exclusively or at high concentration may cause dexamethasone to salt out of the formulation. The amount of tonicity agent will depend on the desired tonicity for the final formulation, but will generally be an amount sufficient to cause the formulations to have an osmolality of about 250-350 mOsm.

The solution formulations optionally contain a nonionic polymer as a viscosity adjusting agent. Many ophthalmically and otically acceptable nonionic polymers are known. These polymers include hydroxyethyl cellulose; hydroxypropylmethyl cellulose; methyl cellulose; carboxymethyl cellulose; polyvinyl pyrrolidone and polyvinyl alcohol. The preferred nonionic polymer is hydroxyethyl cellulose. The nonionic polymer will be present in the formulations of the present invention in an amount of about 0.1-0.5%. In the case of hydroxyethyl cellulose, the preferred concentration of nonionic polymer is 0.2%.

The formulations of the present invention may be prepared without a preservative as a “unit-dose” or “unpreserved” formulation. If a preserved or “multi-dose” formulation is desired, the formulations may contain an ophthalmically and otically acceptable preservative, such as benzyl alcohol or quaternary ammonium halides. Quaternary ammonium preservatives are preferred. Suitable quaternary ammonium preservatives include polyquaternium-1 and benzalkonium halides. Preferred benzalkonium halides are benzalkonium chloride (“BAC”) and benzalkonium bromide. In general, the amount of the preservative ingredient will range from about 0.005-0.3%. In the case where the preservative is BAC, it is preferably present at a concentration of 0.01%. In the case where the preservative is polyquaternium-1, it is preferably present at a concentration of 0.005%.

If desired, a chelating agent may also be added to the formulations of the present invention. Suitable chelating agents include edetate disodium (“EDTA”); edetate trisodium; edetate tetrasodium; and diethyleneamine pentaacetate. Most preferred is EDTA. The chelating agent, if any, will typically be present in an amount from about 0.001-0.1%. In the case of EDTA, the chelating agent is preferably present at a concentration of 0.01%.

In the case of preserved or multi-dose formulations, the solution formulations of the present invention may contain boric acid, as a component of a buffer and/or as a preservative adjunct, typically in an amount from 0.1-1.5%.

The formulations of the present invention will generally have a pH from 5-8, preferably 6.5-7.5. In the case where the antibiotic is ciprofloxacin, however, the preferred pH range is 3-5, and the pH is preferably 4.5. pH can be adjusted with NaOH/HCl. Preferred buffering agents include acetate and citrate buffers. If an acetate buffering system is chosen, the concentration of sodium acetate will generally range from 0.015-0.06%, and will preferably be about 0.03%, and the concentration of acetic acid will generally range from 0.02-0.08, and will preferably be about 0.04%.

The solution formulations of the present invention are intended for topical administration to the eye, ear, nose or skin.

The following examples are intended to illustrate, but not limit, the present invention.

### EXAMPLE 1

The formulations shown in Table 1 were prepared. All ingredient amounts in Table 1 are shown as % (w/v).

The following method was used to prepare Formulation A of Table 1.

1. Tare a 500 mL PYREX media bottle (compound vessel) with stir bar.

2. Weigh out and add to the compounding vessel the specified amount of vitamin E TPGS solution (from 10% w/v stock).

3. Weigh out and add to the vitamin E TPGS solution, the specified amount of glycerin and stir to mix.

4. Weigh out and add dexamethasone powder into the vitamin E TPGS and glycerin solution and allow to dissolve completely by stirring.

5. After dexamethasone is completely dissolved, then weigh out and add with continuous stirring the remaining ingredients in the following order: acetic acid, sodium acetate (trihydrate), ciprofloxacin hydrochloride, disodium edetate (EDTA), boric acid and polyquaternium-1. Allow each ingredient to dissolve completely before proceeding to the next.

6. QS to 90% total batch volume with purified water.

7. Check and adjust pH to 4.5±0.2 with 1N hydrochloric acid and/or 1N sodium hydroxide as needed.

8. QS to 100% total batch volume with purified water and allow to stir until homogenous.

9. Sterile solution formulation is produced either by sterile filtration through a 0.2 micron filter or by autoclaving at 121° C. for approximately 30-35 minutes. Post autoclaved formulation requires stirring to cool down to room temperature and to make a homogenous solution.

TABLE I

| INGREDIENTS | A | B | C | | |
|---|---|---|---|---|---|
| Ciprofloxacin HCl.H2O | 0.35* | 0.35 | 0.35 | | |
| Dexamethasone Alcohol | 0.1 | — | 0.1 | | |
| Dexamethasone sodium | — | 0.132** | — | | |
| phosphate | | | | | |
| Polyquaternium-1 | 0.005 | — | — | | |
| Sodium Acetate | 0.03 | — | 0.03 | | |
| (Trihydrate) | | | | | |
| Acetic Acid | 0.04 | — | 0.04 | | |
| Boric Acid | 0.53 | 1.2 | 0.6 | | |
| Glycerin | 1.25 | 0.82 | — | | |
| Disodium EDTA | 0.01 | 0.01 | 0.01 | | |
| Vitamin E TPGS | 7 | — | — | | |
| Benzalkonium chloride | — | 0.01 | 0.01 | | |
| Hydroxyethylcellulose | — | — | 0.2 | | |
| Sodium Chloride | — | — | 0.53 | | |
| Tyloxapol | — | — | 0.05 | | |
| Polystyrene sulfonic acid, | — | 0.9 | — | | |
| sodium salt | | | | | |
| N-lauroyl sarcosine | — | 0.03 | — | | |
| Hydrochloric Acid and/or | q.s. pH to | q.s. pH to | q.s. pH to | | |
| Sodium Hydroxide | 4.5 ± 0.2 | 5.5 ± 0.2 | 4.5 ± 0.2 | | |
| Purified Water | q.s. to 100 | q.s. to 100 | q.s. to 100 | | |
| Ingredients | D | E | F | G | H |
| Ciprofloxacin HCl.H2O | 0.35 | 0.35 | 0.35 | 0.35 | 0.35 |
| Dexamethasone Alcohol | 0.1 | 0.1 | 0.1 | 0.1 | 0.1 |
| Polyguaternium-1 | 0.005 | 0.005 | 0.005 | — | 0.005 |
| Benzyl Alcohol | — | — | — | 0.9 | — |
| Benzalkonium Chloride | — | — | — | — | — |
| Sodium Acetate | 0.03 | 0.03 | 0.03 | 0.03 | 0.03 |
| (Trihydrate) | | | | | |
| Acetic Acid | 0.04 | 0.04 | 0.04 | 0.04 | 0.04 |
| Sodium Chloride | 0.25 | — | — | — | — |
| Boric Acid | — | 0.53 | 0.53 | 0.53 | 0.53 |
| Glycerin | 1.5 | 1.25 | 1.25 | 1.25 | 1.25 |
| Disodium EDTA | 0.01 | 0.01 | 0.1 | 0.1 | 0.1 |
| Vitamin E TPGS | 5 | 5 | 5 | 5 | 7 |
| Propylene Glycol | — | — | — | — | — |
| Hydrochloric Acid and/or | q.s. pH | q.s. pH | q.s. pH | q.s. pH | q.s. pH |
| Sodium Hydroxide | 4.5 ± 0.2 | 4.5 ± 0.2 | 4.5 ± 0.2 | 4.5 ± 0.2 | 4.5 ± 0.2 |
| Purified Water | q.s. to 100 | q.s. to 100 | q.s. to 100 | q.s. to 100 | q.s. to 100 |
| Ingredients | I | J | K | L | M |
| Ciproiloxacin HCl.H2O | 0.35 | 0.35 | 0.35 | 0.35 | 0.35 |
| Dexamethasone Alcohol | 0.1 | 0.1 | 0.1 | 0.1 | 0.1 |
| Polyguaternium-1 | — | — | — | 0.005 | 0.005 |
| Benzyl Alcohol | 0.9 | 1.0 | 1.0 | — | — |
| Benzalkonium Chloride | — | — | — | — | — |
| Sodium Acetate | 0.03 | 0.03 | 0.03 | 0.03 | 0.03 |
| (Trihydrate) | | | | | |
| Acetic Acid | 0.04 | 0.04 | 0.04 | 0.04 | 0.04 |
| Sodium Chloride | — | — | — | — | — |
| Boric Acid | 0.53 | 1.5 | 1.5 | 1.5 | 1.5 |
| Glycerin | 1.25 | — | — | — | — |
| Disodium EDTA | 0.1 | 0.1 | 0.1 | 0.1 | 0.1 |
| Vitamin E TPGS | 7 | 5 | 7 | 5 | 7 |
| Propylene Glycol | — | 10 | 10 | 10 | 10 |
| Hydrochloric Acid and/or | q.s. pH | q.s. pH | q.s. pH | q.s. pH | q.s. pH |
| Sodium Hydroxide | 4.5 ± 0.2 | 4.5 ± 0.2 | 4.5 ± 0.2 | 4.5 ± 0.2 | 4.5 ± 0.2 |
| Purified Water | q.s. to 100 | q.s. to 100 | q.s. to 100 | q.s. to 100 | q.s. to 100 |
| INGREDIENTS | N | O | P | Q | |
| Ciprofloxacin HCl.H2O | 0.35 | 0.35 | 0.35 | 0.35 | |
| Dexamethasone Alcohol | 0.1 | 0.1 | 0.1 | 0.1 | |
| Polyguaternium-1 | — | — | 0.005 | 0.005 | |
| Sodium Citrate (dihydrate) | — | — | 0.0425 | 0.0425 | |
| Citric Acid | 0.01 | 0.01 | 0.0175 | 0.0175 | |
| Boric Acid | 0.6 | 0.6 | — | 0.53 | |
| Sodium Chloride | 0.25 | 0.25 | 0.25 | 0.25 | |
| Glycerin | — | — | 1.5 | 1.25 | |
| Disodium EDTA | — | — | 0.01 | 0.01 | |
| Vitamin E TPGS | 10 | 40 | 5 | 7 | |
| Hydrochloric Acid and/or | 0.01 | 0.01 | q.s. pH to | q.s. pH to | |
| Sodium Hydroxide | | | 4.5 ± 0.2 | 4.5 ± 0.2 | |
| Purified Water | — | — | q.s. to 100 | q.s. to 100 | |

*equivalent to 0.3% Ciprofloxacin base
**equivalent to 0.1% Dexamethasone alcohol

### EXAMPLE 2

Formulations A and D were evaluated to determine whether they met the preservative effectiveness standards of the U.S. and European Pharmacopeia. The results are shown in Table 2.

TABLE 2

| Ingredients | Formulation A | Formulation D |
|---|---|---|
| Ciprofloxacin HCl.H2O | 0.35 | 0.35 |
| Dexamethasone Alcohol | 0.1 | 0.1 |
| Polyguaternium-1 | 0.005 | 0.005 |
| Sodium Acetate | 0.03 | 0.03 |
| Trihydrate | | |
| Acetic Acid | 0.04 | 0.04 |
| Boric Acid | 0.53 | — |
| Glycerin | 1.25 | 1.5 |
| Disodium EDTA | 0.01 | 0.01 |
| Vitamin E TPGS | 7 | 5 |
| Hydrochloric Acid and/or | Adjust pH to 4.5 ± 0.2 | Adjust pH to 4.5 ± 0.2 |
| Sodium Hydroxide | | |
| Purified Water | q.s. to 100 | q.s. to 100 |
| Preservative | Passed USP* | Failed USP |
| Effectiveness | | |
| Test Results | And Ph. Eur B** | |

*U.S. Pharmacopeia preservative standards for ophthalmic preparations
**Minimum European Pharmacopeia standards for ophthalmic preparations

### EXAMPLE 3

The turbidity of each of Formulations A, E, F and G was measured using a turbidimeter. The results are shown in Table 3.

TABLE 3

| INGREDIENTS | A | E | F | G |
|---|---|---|---|---|
| Ciprofloxacin HCl.H2O | 0.35 | 0.35 | 0.35 | 0.35 |
| Dexamethasone Alcohol | 0.1 | 0.1 | 0.1 | 0.1 |
| Polyquaternium-1 | 0.005 | 0.005 | 0.005 | — |
| Sodium Acetate (Trihydrate) | 0.03 | 0.03 | 0.03 | 0.03 |
| Acetic Acid | 0.04 | 0.04 | 0.04 | 0.04 |
| Boric Acid | 0.53 | 0.53 | 0.53 | 0.53 |
| Glycerin | 1.25 | 1.25 | 1.25 | 1.25 |
| Disodium EDTA | 0.01 | 0.01 | 0.1 | 0.1 |
| Vitamin E TPGS | 7 | 5 | 5 | 5 |
| Benzyl Alcohol | — | — | — | 0.9 |
| Hydrochloric Acid and/or Sodium | q.s. pH to | q.s. pH to | q.s. pH to | q.s. pH to |
| Hydroxide | 4.5 ± 0.2 | 4.5 ± 0.2 | 4.5 ± 0.2 | 4.5 ± 0.2 |
| Purified Water | q.s. to 100 | q.s. to 100 | q.s. to 100 | q.s. to 100 |
| Turbidity (NTU) pre-autoclave | 6.0, 6.1, 6.3 | 6.7, 6.5, 6.7 | 9.1, 9.2, 9.2 | 130, 130, 132 |
| Turbidity (NTU) post-autoclave | 6.2, 6.3, 6.4 | 6.6, 6.8, 6.7 | 5.75, 5.88, 5.8 | 6.05, 6.21, 5.75 |

### EXAMPLE 4

The anti-inflammatory activity of each of Formulations A, B and C was evaluated in a mouse model of external ear canal inflammation. Wright, et al., “An Animal Model for External Ear Canal Inflammation,” Laryngoscope, 110(7):1112-8 (2000). Each of the formulations was tested in four mice. A single application of 2.5 micrograms of tetradecanoylphorbol acetate (TPA) in 20 microliters of acetone was made to both ear canals of each animal. Twenty microliters of the formulation to be tested were then immediately applied to the left ear canal, with the right ear serving as a TPA-only control. Approximately six hours later a second dose of the tested formulation was applied, followed by two additional doses at 24 and 32 hours respectively. The animals were sacrificed at 48 hours after the initial TPA application and ear canal tissue specimens were taken for histological analysis.

Tissue specimens were fixed in 2.5%, phosphate-buffered glutaraldehyde and processed for embedding in plastic resin. A series of at least 75 sections (2 to 5 microns thick) were then cut from each specimen. Following toluidin blue staining, all sections were studied by light microscopy. In order to obtain a quantitative estimate of the severity of inflammation, a representative section was chose from each series and all polymorphonuclear leucocytes (PMNs) in a 50× microscopic field within that section were counted. A higher number of PMNs indicates a greater degree of inflammation. The results appear in Table 4.

TABLE 4

| | | Animal | | | | |
|---|---|---|---|---|---|---|
| Formulation | | 1 | 2 | 3 | 4 | Average |
| A | Right ear-control | 135 | 49 | 55 | 125 | 91 |
| | (TPA alone) | | | | | |
| A | Left ear | 2 | 0 | 3 | 4 | 2.25 |
| B | Right-ear-control | 74 | 69 | 86 | 81 | 77.5 |
| | TPA alone | | | | | |
| B | Left ear | 52 | 61 | 81 | 69 | 65.75 |
| C | Right-ear-control | 70 | 64 | 112 | 103 | 87.25 |
| | TPA alone | | | | | |
| C | Left ear | 16 | 2 | 24 | 103 | 10.75 |

All four of the animals on which Formulation A was tested showed dramatic reduction of the TPA-induced inflammation. In addition to marked reduction of epidermal hyperplasia and dermal swelling there was almost total block of PMN infiltration as indicated in Table 4.

In contrast, Formulation B appeared to have little effect on the inflammatory response. Thickening of the EAC tissues was little changed by four applications of the tested formulation and, as shown in Table 4, there was only a very marginal effect on PMN infiltration of the ear canal tissues. As in the case of Formulation A, animals treated with Formulation C showed a dramatic reduction of dermal swelling and epidermal hyperplasia as well as effective blocking of PMN infiltration following treatment with the tested formulation.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

What is claimed is:

1. A topically administrable solution composition intended for application to the eye, ear, nose or skin comprising
a) 0.01-1% (w/v) of a corticosteroid;
b) 0.1-1.5% (w/v) of an antibiotic drug;
c) a vitamin E tocopheryl derivative in an amount sufficient to solubilize the corticosteroid;
d) a tonicity agent in an amount sufficient to cause the composition to have an osmolality of about 250-350 mOsm; and
e) a buffering agent.

2. A topically administrable solution composition intended for application to the eye, ear, nose or skin comprising
a) 0.01-0.5% (w/v) of a corticosteroid selected from the group consisting of dexamethasone alcohol and dexamethasone acetate;
b) 0.1-1% (w/v) of a fluoroquinolone antibiotic drug;
c) a vitamin E tocopheryl derivative in an amount sufficient to solubilize the corticosteroid;
d) a tonicity agent in an amount sufficient to cause the composition to have an osmolality of about 250-350 mOsm; and
e) a buffering agent.

3. The composition of claim 2 wherein the fluoroquinolone antibiotic drug is selected from the group consisting of ciprofloxacin, moxifloxacin, gatifloxacin, gemifloxacin, norfloxacin, ofloxacin, levofloxacin and trovofloxacin.

4. The composition of claim 3 wherein the fluoroquinolone antibiotic drug is ciprofloxacin.

5. The composition of claim 4 wherein the corticosteriod is dexamethasone alcohol and is present in a concentration of 0.1% (w/v) and the fluoroquinolone antibiotic drug is present in a concentration of 0.3% (w/v).

6. The composition of claim 1 wherein the composition further comprises a nonionic polymer selected from the group consisting of hydroxyethyl cellulose; hydroxypropylmethyl cellulose; methyl cellulose; carboxymethyl cellulose; polyvinyl pyrrolidone and polyvinyl alcohol.

7. The composition of claim 1 wherein the composition further comprises a preservative.

8. The composition of claim 7 wherein the preservative is a quaternary ammonium preservative in an amount from 0.005-0.3% (w/v).

9. The composition of claim 8 wherein the composition further comprises 0.001-0.1% (w/v) of a chelating agent and 0.1-1.5% (w/v) of boric acid.

10. The composition of claim 9 wherein the quaternary ammonium preservative is selected from the group consisting of polyquaternium-1 and benzalkonium halides; and the chelating agent is selected from the group consisting of edetate disodium; edetate trisodium; edetate tetrasodium; and diethyleneamine pentaacetate.

11. The composition of claim 10 wherein the quaternary ammonium preservative is benzalkonium chloride and the chelating agent is edetate disodium.

12. A topically administrable solution composition intended for application to the eye, ear, nose or skin consisting essentially of
a) 0.1% (w/v) dexamethasone alcohol;
b) 0.35% (w/v) ciprofloxacin hydrochloride, monohydrate;
c) 7% (w/v) α-tocopheryl polyoxyethylene glycol (1000) succinate;
d) a tonicity agent in an amount sufficient to cause the composition to have an osmolality of about 250-350 mOsm;
e) a buffer comprising sodium acetate and acetic acid;
f) 0.005% (w/v) polyquaternium-1;
g) 0.01% (w/v) edetate disodium;
h) 0.4-0.6% (w/v) boric acid; and
wherein the composition has a pH from 4.3-4.7.
